(19) United States
(12) Patent Application Publication
(10) Pub. No.: US 2007/0259040 A1
(43) Pub. Date: Nov. 8, 2007

(54) Novel triptan formulations and methods for making them

(76) Inventor: S. Rao Cherukuri, Vienna, VA (US)

Correspondence Address: THORPE NORTH & WESTERN, LLP., 8180 SOUTH 700 EAST, SUITE 350, SANDY, UT, 84070, US

(21) Appl. No.: 11/799,751

(22) Filed: May 1, 2007

Related U.S. Application Data
(60) Provisional application No. 60/796,789, filed on May 1, 2006.

(51) Int. Cl. A61K 31/787; A61K 31/4184; A61K 9/20

(52) U.S. Cl. 424/464; 514/394; 424/78.14

(57) ABSTRACT

Rapidly disintegrating oral triptan formulations having superior palatability and methods of making such are provided herein. A rapidly disintegrating oral triptan composition can comprise a triptan compound, a resin, a lubricant, a disintegrant, and a compressible material, where the triptan is admixed with the resin forming a taste-masked triptan composition, which is further admixed with the lubricant, the disintegrant, and the compressible material to form the rapidly disintegrating oral triptan composition.

### RELATED APPLICATIONS

This application claims the benefit of U. S. Provisional Application Ser. No. 60/796,789 filed on May 1, 2006, which is incorporated herein by reference.

### FIELD OF THE INVENTION

The present invention relates to orally disintegrating tablets and a method for their manufacture. More particularly, the present invention relates to an orally disintegrating tablet made up of rapidly dispersing triptan compositions.

### BACKGROUND OF THE INVENTION

Pharmaceutical compositions may be produced in a variety of dosage forms, depending upon the desired route of administration of the therapeutic material. Oral dosage forms, for example, include such solid compositions as tablets, emulsions, and suspensions. The particular dosage form utilized will depend on such factors as the solubility and chemical reactivity of the pharmaceutical active. Further, the dosage form may be selected so as to optimize delivery of the pharmaceutical active and/or consumer acceptability of the composition.

Tablet compositions offer many advantages, including ease of product handling, chemical and physical stability, portability (in particular, allowing ready availability to the consumer when needed), aesthetic acceptability and dosage precision, i.e., ensuring consistent and accurate dosages of the pharmaceutical active. However, liquid formulations may offer advantages in the treatment of certain disorders, such as disorders of the upper gastrointestinal tract, wherein delivery of an active material dissolved or dispersed in a liquid ensures rapid and complete delivery to the afflicted area. In an effort to obtain the therapeutic advantages associated with liquid formulations as well as the broad advantages associated with solids, many chewable tablet formulations have been developed.

One important factor in formulating chewable tablets is palatability and mouth feel, especially in tablets that include pharmaceutical dosages. Many pharmaceutical and confectionery tablets are designed to be chewed either to provide proper flavor or to increase the surface area of a particular drug to permit rapid activity in the digestive tract or circulatory systems. However, many pharmaceutical ingredients usually have both an unpleasant mouth feel and unpalatable taste due to chalkiness, grittiness, dryness, and astringent properties of these materials. Accordingly, the practical value of these materials is substantially diminished since patients finding them objectionable may fail to take them as prescribed. As such, formulations continue to be investigated to ease the mouth feel and palatability of such compositions.

### SUMMARY OF THE INVENTION

Novel rapidly disintegrating oral triptan formulations having superior palatability and methods of making such are provided herein. These formulations provide easy dosage form administration and consumer convenience and compliance, fast onset of therapeutic activity combined with substantially complete disintegration of the formulation in less than about one minute.

In one embodiment, a rapidly disintegrating oral triptan composition can comprise a triptan compound, a resin, a lubricant, a disintegrant, and a compressible material, where the triptan is admixed with the resin forming a taste-masked triptan composition, which is further admixed with the lubricant, the disintegrant, and the compressible material to form the rapidly disintegrating oral triptan composition.

In another embodiment, a method of making a rapidly disintegrating oral triptan composition can comprise the steps of admixing a resin and a triptan compound forming a taste-masked triptan compound, and admixing the taste-masked triptan compound with a lubricant, a disintegrant, and a compressible material forming a rapidly disintegrating oral triptan composition.

In still another embodiment, a method of administering a triptan composition to a subject can comprise the steps of providing the triptan composition in a rapidly disintegrating oral dosage form, where the triptan composition includes a triptan compound, a resin, a lubricant, a disintegrant, and a compressible material, and administering the rapidly disintegrating oral dosage form to the subject's oral cavity. The triptan can be admixed with the resin forming a taste-masked triptan composition, where the taste-masked triptan composition can be further admixed with the lubricant, the disintegrant, and the compressible material to form a rapidly disintegrating oral triptan composition.

In one aspect, a triptan composition for oral administration to a subject can comprise a triptan compound in combination with a resin which allows the triptan composition to substantially dissolve in the oral cavity and masks an unpleasant taste of the triptan compound.

In another aspect, a method of masking an unpleasant taste of a triptan compound to be administered in an oral dosage form to the oral cavity of a subject can comprise combining the triptan compound with a resin, which allows the triptan composition to substantially dissolve in the oral cavity and mask the unpleasant taste of the triptan compound.

In another aspect, a method of making an oral dissolvable triptan composition in an oral dosage form can comprise combining a triptan compound with a resin, which allows the triptan composition to substantially dissolve in the oral cavity and mask an unpleasant taste of the triptan compound.

In another aspect, a method of administering an oral dissolvable triptan composition to a subject can comprise providing the triptan composition as previously recited; and administering the oral dosage form to the subject's oral cavity.

In another aspect, the triptan compound can be selected from the group consisting of sumatriptan, eletriptan, rizatriptan, frovatriptan, almotriptan, zolmitriptan, naratriptan, pharmaceutically acceptable salt or hydrate thereof, and mixtures thereof.

In another aspect, the triptan compound can be present in an amount from about 0.1 wt % to about 50 wt %.

In another aspect, the resin can be selected from the group consisting of cholestyramine resin, cationic resins including copolymers of methacrylic acid crosslinked with divinylbenzene, sodium polystyrene sulfonate, polacrilin potassium and mixtures thereof.

In another aspect, the resin can be present in an amount from about 20 wt % to about 95 wt %.

In another aspect, the triptan composition can further comprise an overcoating, wherein said overcoating is provided by a coating agent selected from the group consisting of a polymer, a fat, a wax, an emulsifier, and mixtures thereof.

In another aspect, the coating agent can be present in amount from about 0.1 wt % to about 50 wt %.

In another aspect, the polymer can be selected from the group consisting of cellulosic polymers including methylcellulose (MC), carboxymethylcellulose (CMC), hydroxypropylcellulose (HPC), hydroxylpropylmethylcellulose (HPMC), hydroxyethylcellulose (HEC); vinyl polymers including polyvinylpyrrolidone (PVP), polyvinyl alcohol (PVA); acrylic polymers and copolymers including acrylic acid polymer, methacrylic acid copolymers, ethyl acrylate-methyl methacrylate copolymers; and mixtures thereof; the emulsifier is selected from the group consisting of alkyl aryl sulfonates, alkyl sulfates, sulfonated amides and amines, sulfated and sulfonated esters and ethers, alkyl sulfonates, polyethoxylated esters, monoglycerides, diglycerides, triglycerides, diacetyl tartaric esters of monoglycerides, polyethyleneglycol esters, polyglycerol esters, sorbitan esters and ethoxylates, lactylated esters, phospholipids such as lecithin, polyoxyethylene sorbitan esters, propylene glycol esters, sucrose esters, and mixtures thereof; and the fat and/or wax is individually selected from the group consisting of bees wax, carnuba wax, spermaceti, mineral oil, paraffin, microcrystalline wax, polyethylene wax and mixtures thereof.

In another aspect, the triptan composition can further comprise a lubricant and a disintegrant, wherein the lubricant is selected from the group consisting of magnesium stearate, zinc stearate, calcium stearate, stearic acid, glyceryl palmitostearate, vegetable oils, polyethylene glycols, polyvinyl alcohols, talc, sodium benzoate, sodium stearyl fumarate, magnesium oxide, poloxamer, sodium lauryl sulphate, polyoxy ethylene monostearate, cocoa butter, hydrogenated tallow, hydrogenated cotton seed oil, canola oil, palm kernel oil, soybean oil, stannol esters, hydrogenated palm kernel oil, mineral oil, cocoa butter, cocoa butter substitutes, polysaccharides, and mixtures thereof, and the disintegrant is selected from the group consisting of mannitol, alginic acid, carboxymethylcellulose, hydroxypropylcellulose, microcrystalline cellulose, colloidal silicon dioxide, croscarmellose sodium, crospovidone, magnesium aluminum silicate, methylcellulose, povidone, sodium alginate, sodium starch glycolate, and mixtures thereof.

In another aspect, the lubricant can be present in an amount from about 0.1 wt % to about 30 wt % and the disintegrant is present in an amount from about 1 wt % to about 75 wt %.

In another aspect, the triptan composition can further comprise a taste-masking agent selected from the group consisting of volatile oils, synthetic flavor oils, flavoring aromatics, oils, liquids, oleoresins, extracts, and mixtures thereof.

In another aspect, the taste-masking agent can present in an amount from about 0.1 wt % to about 30 wt %.

In another aspect, the oral dosage form can be selected from the group consisting of tablets, capsules, caplets, powders, pellets, granules, chews, beads, liquid suspensions, and sprays.

In another aspect, the triptan compound can be complexed to the resin through a mechanism selected from the group consisting of chelation, coordination, ionic bonding, covalent bonding, coordinate-covalent bonding, and combinations thereof.

In another aspect, the oral dosage form can be formulated for controlled release.

In another aspect, the oral dosage form can be a rapidly disintegrating oral dosage form.

In another aspect, the rapidly disintegrating oral dosage form can dissolve in an oral cavity in less than about 45 seconds.

In another aspect, the rapidly disintegrating oral dosage form can dissolve in an oral cavity in less than about 30 seconds.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

### BRIEF DESCRIPTION OF THE DRAWINGS

Additional features and advantages of the invention will be apparent from the detailed description which follows, taken in conjunction with the accompanying drawings, which together illustrate, by way of example, features of the invention; and, wherein:

FIG. 1 is a graphical representation of a dissolution profile of a rapidly disintegrating triptan in accordance with an embodiment of the present invention at pH 1.2 compared to a commercially available triptan composition (IMITREX 25 mg immediate release tablets);

FIG. 2 is a graphical representation of a dissolution profile of a rapidly disintegrating triptan in accordance with an embodiment of the present invention at pH 4.5 compared to a commercially available triptan composition (IMITREX 25 mg immediate release tablets); and

FIG. 3 is a graphical representation of a dissolution profile of a rapidly disintegrating triptan in accordance with an embodiment of the present invention at pH 6.8 compared to a commercially available triptan composition (IMITREX 25 mg immediate release tablets).

Reference will now be made to the exemplary embodiments illustrated, and specific language will be used herein to describe the same. It will nevertheless be understood that no limitation of the scope of the invention is thereby intended.

### DETAILED DESCRIPTION OF THE INVENTION

Before the present invention is disclosed and described, it is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

It must be noted that, as used in this specification and the appended claims, the singular forms “a,” “an,” and, “the” include plural referents unless the context clearly dictates otherwise. Thus, for example, reference to “a granule” includes one or more of such devices, reference to “an amount of a polysaccharide” includes reference to one or more amounts of polysaccharides, and reference to “the binder” includes reference to one or more binders.

Definitions

In describing and claiming the present invention, the following terminology will be used in accordance with the definitions set forth below.

As used herein, “triptan” or “triptan compound” refers to a family of tryptamine drugs typically used as abortive medication in the treatment of migraine and cluster headaches. Generally, their action is attributed to their binding to serotonin 5-HT_{1B} and 5-HT_{1D} receptors in cranial blood vessels (causing their constriction) and subsequent inhibition of pro-inflammatory neuropeptide release. As such, this terms includes any tryptamine compounds providing such an effect including, but not limited to, sumatriptan, eletriptan, rizatriptan, frovatriptan, almotriptan, zolmitriptan, naratriptan, pharmaceutically acceptable salts thereof, hydrates thereof, and mixtures thereof

As used herein, the term “sugar alcohol” refers to a hydrogenated form of carbohydrate, whose carbonyl group (aldehyde or ketone, reducing sugar) has been reduced to a primary or secondary hydroxyl group. This term is also commonly known as polyol, polyhydric alcohol, or polyalcohol. Sugar alcohols include, but are not limited to, arabitol, erythritol, hydrogenated starch hydrolysates, isomalt, lactitol, maltitol, mannitol, sorbitol, xylitol, galactitol, inositol, ribitol, dithioerythritol, dithiothreitol, glycerol, and mixtures thereof.

As used herein, “sugar” refers to monosaccharides and disaccharides, as well as other carbohydrates. As used herein, the term “carbohydrate” refers to molecules having straight-chain aldehydes or ketones with many hydroxyl groups added, usually one on each carbon atom that is not part of the aldehyde or ketone functional group. Carbohydrates include monosaccharides, disaccharides, oligosaccharides, and polysaccharides. Carbohydrates are the most abundant biological molecules, and fill numerous roles in living things, such as the storage and transport of energy (starch, glycogen) and structural components (cellulose in plants, chitin in animals).

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients.

As used herein, “admixed” means that the drug and/or other ingredients can be dissolved, dispersed, or suspended in the carrier. In some cases, the drug may be uniformly admixed in the carrier.

As used herein, “subject” refers to a mammal that may benefit from the administration of a drug composition or method of this invention. Examples of subjects include humans, and may also include other animals such as horses, pigs, cattle, dogs, cats, rabbits, and aquatic mammals.

As used herein, “pharmaceutically acceptable” refers to any inert and pharmaceutically acceptable material that has substantially no biological activity, and makes up a substantial part of the formulation.

As used herein, “oral dosage form” and the like refers to a formulation that is ready for administration to a subject through the oral route of administration. Examples of known oral dosage forms include, without limitation, tablets, capsules, caplets, powders, pellets, granules, chews, liquid dispersions, beads, sprays etc. In some aspects, powders, pellets, and granules may be coated with a suitable polymer or a conventional coating material to achieve, for example, greater stability in the gastrointestinal tract, or to achieve the desired rate of release. Moreover, capsules containing a powder, pellets, or granules may be further coated. Tablets and caplets may be scored to facilitate division of dosing. Alternatively, the dosage forms of the present invention may be unit dosage forms wherein the dosage form is intended to deliver one therapeutic dose per administration.

As used herein, “controlled release” refers to the drug release that is different from an immediate release. Typically, in an immediate release dosage form, about more than 80% of the drug is released from the dosage form in vitro within about 2 hrs. This release may be measured in terms of dissolution of the drug in the dissolution medium. In one aspect, the release can be measured under USP conditions, i.e., where the pH is maintained at 1.2 for 2 hours, followed by a pH of 6.8 for the rest of the time. In another aspect, the release can be measured at a pH of 1.2 for the entire period of measurement. Examples of such controlled release include sustained release, slow-release, delayed-release, pulsatile release etc., which terms are generally known in the art and to the extent they mean a release other than an immediate release.

As used herein, the term “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still contain such an item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a defacto equivalent of any other member of the same list solely based on their presentation in a common group without indications to the contrary.

As used herein, “%” refers to the weight percent of a compound as it relates to the overall composition unless otherwise indicated.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 micron to about 5 microns” should be interpreted to include not only the explicitly recited values of about 1 micron to about 5 microns, but also include individual values and sub-ranges within the indicated range. Thus, included in this numerical range are individual values such as 2, 3.5, and 4 and sub-ranges such as from 1-3, from 2-4, and from 3-5, etc.

This same principle applies to ranges reciting only one numerical value. Furthermore, such an interpretation should apply regardless of the breadth of the range or the characteristics being described.

The Invention

The present invention provides compositions and methods for rapidly disintegrating oral dosage forms of triptan. It is noted that when discussing a triptan composition or a method involving a triptan, each of these discussions can be considered applicable to each of these embodiments, whether or not they are explicitly discussed in the context of that embodiment. Thus, for example, in discussing the resins present in a triptan composition, those resins can also be used in a method for making triptan composition, and vice versa.

In one embodiment, a rapidly disintegrating oral triptan composition can comprise a triptan compound, a resin, a lubricant, a disintegrant, and a compressible material, where the triptan is admixed with the resin forming a taste-masked triptan composition, which is further admixed with the lubricant, the disintegrant, and the compressible material to form the rapidly disintegrating oral triptan composition.

In another embodiment, a method of making a rapidly disintegrating oral triptan composition can comprise the steps of admixing a resin and a triptan compound forming a taste-masked triptan compound, and admixing the taste-masked triptan compound with a lubricant, a disintegrant, and a compressible material forming a rapidly disintegrating oral triptan composition.

In still another embodiment, a method of administering a triptan composition to a subject can comprise the steps of providing the triptan composition in a rapidly disintegrating oral dosage form, where the triptan composition includes a triptan compound, a resin, a lubricant, a disintegrant, and a compressible material, and administering the rapidly disintegrating oral dosage form to the subject's oral cavity. The triptan can be admixed with the resin forming a taste-masked triptan composition, where the taste-masked triptan composition can be further admixed with the lubricant, the disintegrant, and the compressible material to form a rapidly disintegrating oral triptan composition.

In yet another embodiment relates to a tablet, which is compressed into a predetermined shape after blending the granulated material with a suitable lubricant. An ideal orally disintegrating tablet formulation rapidly disintegrates in the buccal cavity dispersing the contents and has a pleasant taste and smooth creamy mouthfeel (no grittiness), and complete drug release occurs in the gastrointestinal tract so as to be bioequivalent to the reference immediate release product. This convenience leads to better compliance with dosing regimen and as a consequence, to enhanced therapy. From an industrial/commercial utility point of view, the tablets should have sufficient strength to be suitable for packaging in HDPE bottles and push-through blisters for storage, transportation, and distribution.

Some prior disclosures include: U.S. Pat. Nos. 4,305,502, 4,371,516, and 5,738,875; 5,178,878, 6,269,615 and 6,221,392; 5,464,632 and 6,106,861; 5,720,974; 5,576,014; and 6,514,492; and WO 2002085336 A1. All of these publications are hereby incorporated by reference.

In one aspect, the triptan is selected from the group consisting of: sumatriptan, eletriptan, rizatriptan, frovatriptan, almotriptan, zolmitriptan, and naratriptan. These actives may be administered in a dosage from about 0.01 to about 300 mg, or from about 1 mg to about 200 mg, or about 5 mg to 100 mg or about 10 mg to about 150 mg. The triptan can be present in an amount from about 0. 1 wt % to about 50 wt %. In some aspects the triptan may range from about 0.1% to about 25%; about 0.1% to about 10%; about 1% to about 30%; about 1% to about 20%; about 1% to about 10%; about 5% to about 30%; about 5% to about 20%; about 10% to about 30%; about 10% to about 20%; about 15% to about 30%; or about 20% to about 40%.

In one aspect, the triptan composition prepared according to the invention herein disintegrates rapidly, in about sixty seconds or less; or in about 45 seconds or less; or in about 30 seconds or less; or in about 20 seconds or less.

In a specific aspect, the triptan is taste-masked with a composition comprising a resin. The resin composition may include a resin such as Amberlite or Duolite or their equivalents, or a mixture thereof. This resin-taste-masked composition may be used directly for administration to a patient in need thereof. Alternatively, the resin-taste-masked composition may be further processed into formulations such as tablets, capsules, caplets, granules, sachets, etc. Such processing may include additional taste-masking or some other coating as needed. Thus, in one aspect, the composition of the present invention is substantially taste-masked with a resin. In another aspect, the composition of the present invention is taste-masked solely with a resin. In yet another aspect, the composition is taste-masked with both a resin and a non-resin taste-masking material. In yet another aspect, the composition is first taste-masked with a resin and optionally followed with another taste-masking with a non-resin composition.

Ion Exchange Resins Useful in the Practice of the Present Invention Ion exchange resins useful in the practice of the present invention include, but are not limited to, anionic resins such as: DUOLITE AP143/1083 (cholestyramine resin USP) and cationic resins such as: AMBERLITE IRP-64 (a porous copolymers of methacrylic acid crosslinked with divinylbenzene), AMBERLITE IRP-69 (Sodium polystyrene sulfonate USP) and AMBERLITE IRP-88 (Polacrilin Potassium). The DUOLITE and AMBERLITE resins are available from the Rohm and Haas Company, Philadelphia, PA. The DOWEX resins, available from the Dow Chemical Company, Midland, MI, are also useful in the practice of the present invention. These resins are available with variable crosslinkings and in a variety of particle sizes.

The amount of resin needed for taste-masking varies from about 20% to about 95%. In some aspects, the resin may range from: about 30% to about 90%; about 40% to about 90%; about 50% to about 90%; about 60% to about 90%; about 70% to about 90%; about 20% to about 75%; about 30% to about 80%; about 30% to about 70%; about 40% to about 90%; about 40% to about 80%; about 40% to about 70%.

Lubricants that may be used in accordance with embodiments of the present invention include, but are not limited to, magnesium stearate, zinc stearate, calcium stearate, stearic acid, glyceryl palmitostearate, vegetable oils, polyethylene glycols, polyvinyl alcohols, talc, sodium benzoate, sodium stearyl fumarate, magnesium oxide, poloxamer, sodium lauryl sulphate, polyoxy ethylene monostearate, cocoa butter, hydrogenated tallow, hydrogenated cotton seed oil, canola oil, palm kernel oil, soybean oil, stannol esters, hydrogenated palm kernel oil, mineral oil, cocoa butter, cocoa butter substitutes, polysaccharides, and mixtures thereof.

The amount of lubricant that may be present varies from about 0.1% to about 30%. In some aspects, the lubricant may range from about 1% to about 30%; about 5% to about 30%; about 10% to about 30%; about 0.1% to about 20%; about 0.1% to about 10%; about 1% to about 20%; about 1% to about 10%; about 5% to about 25%; about 5% to about 15%; or about 5% to about 10%.

Disintegrants that may be used in accordance with embodiments of the present invention include, but are not limited to, mannitol, alginic acid, carboxymethylcellulose, hydroxypropylcellulose, microcrystalline cellulose, colloidal silicon dioxide, croscarmellose sodium, crospovidone, magnesium aluminum silicate, methylcellulose, povidone, sodium alginate, sodium starch glycolate, and starch, as well as other conventional disintegrants well known to persons skilled in the art.

The amount of disintegrant that may be present varies from about 1% to about 75%. In some aspects, the disintegrant may range from about 1% to about 50%; about 1% to about 30%; about 10% to about 75%; about 10% to about 50%; about 20% to about 75%; about 20% to about 50%; about 30% to about 75%; about 30% to about 50%; about 25% to about 75%; or about 25% to about 50%.

Compressible materials that may be used in accordance with embodiments of the present invention include, but are not limited to, sugars, a sugar product such “Di-Pac” from the Domino Sugar Corp., a dextrose such as “Cantab” from Compton Knowles Inc., or other compressible sugar materials such as monosaccharides, disaccharides, oligosaccharides and polysaccharides. If, on the other hand, the encapsulated product is to be sugar-free, then examples of the compressible material can be sugar alcohols. Sugar-free materials include, without limitation, arabitol, erythritol, hydrogenated starch hydrolysates, isomalt, lactitol, maltitol, mannitol, sorbitol, xylitol, galactitol, inositol, ribitol, dithioerythritol, dithiothreitol, glycerol, calcium phosphates, microcrystalline celluloses, polydextrose, erythritols, other compressible materials and mixtures thereof. In one embodiment, the compressible material is sorbitol. In another embodiment, the compressible material is mannitol. In yet another embodiment, the compressible material is a cellulose.

The amount of compressible material that may be present varies from about 1% to about 75%. In some aspects, the compressible material may range from about 1% to about 50%; about 1% to about 30%; about 10% to about 75%; about 10% to about 50%; about 20% to about 75%; about 20%.to about 50%; about 30% to about 75%; about 30% to about 50%; about 25% to about 75%; or about 25% to about 50%.

Plasticizers that may be used in accordance with embodiments of the present invention include, but are not limited to, lanolin, mineral oil, petrolatum, benzyl phenylformate, chlorobutanol, diethyl phthalate, glycerol, polyethylene glycol, propylene glycol, sorbitol, triacetin, mixtures thereof, as well as other conventional plasticizers well known to persons skilled in the art.

Additional taste-masking agents that may be used in accordance with embodiments of the present invention include polymers, fats, waxes, emulsifiers, flavors, colors and sweeteners. In one embodiment, the taste-masking agents may be selected from the group consisting of polymer, fats, waxes, emulsifiers, and mixtures thereof. The use of particular polymers, fats, emulsifiers, or waxes may allow the product of the present inventive subject matter, whether encapsulated or unencapsulated, to provide controlled release of the triptan compound. Although not being bound by any particular theory, it is thought that the controlled release occurs due to the entrapment of the triptan compound in the particular polymer, fat, emulsifier, and/or wax.

Flavors may be chosen from natural and synthetic flavor liquids. Flavors useful in the present inventive compositions include, but are not limited to, volatile oils, synthetic flavor oils, flavoring aromatics, oils, liquids, oleoresins or extracts derived from plants, leaves, flowers, fruits, stems and combinations thereof. A non-limiting list of examples include citrus oils such as lemon, orange, grape, lime and grapefruit and fruit essences including apple, pear, peach, grape, strawberry, raspberry, cherry, plum, pineapple, apricot or other fruit flavors. Other useful flavorings include aldehydes and esters such as benzaldehyde (cherry, almond), citral, i.e., alphacitral (lemon, lime), neral, i.e., betal-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), and 2-dodecenal (citrus, mandarin), and mixtures thereof. Further examples of flavors useful in the inventive compositions include, without limitation, beef flavorings, chicken flavorings, rice flavorings, lamb flavorings, pork flavorings, seafood flavorings, and mixtures thereof. The sweeteners may be chosen from the following non-limiting list: flucose (corn syrup), dextrose, invert sugar, fructose, and mixtures thereof; saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; sugar alcohols such as sorbitol, mannitol, zylitol, and the like. Also contemplated are hydrogenated starch hydrolysates and synthetic sweetener 3,6-dihydro-6-methyl-1-1-1 ,2,3-oxath-iazin-4- one-2,2-dioxide, particularly the potassium salt (acesulfame-K) and sodium and calcium salts thereof. Other sweeteners may also be used.

Emulsifiers that may be used in accordance with embodiments of the present invention include, but are not limited to, alkyl aryl sulfonates, alkyl sulfates, sulfonated amides and amines, sulfated and sulfonated esters and ethers, alkyl sulfonates, polyethoxylated esters, monoglycerides, diglycerides, triglycerides, diacetyl tartaric esters of monoglycerides, polyethyleneglycol esters, polyglycerol esters, sorbitan esters and ethoxylates, lactylated esters, phospholipids such as lecithin, polyoxyethylene sorbitan esters, propylene glycol esters, sucrose esters, and mixtures thereof. The emulsifier may be either saturated or unsaturated.

Polymers that may be used in accordance with embodiments of the present invention include, but are not limited to, cellulosic polymers, such as methylcellulose (MC), carboxymethylcellulose (CMC), hydroxypropylcellulose (HPC), hydroxylpropylmethylcellulose (HPMC), hydroxyethylcellulose (HEC), and the like; vinyl polymers, such as polyvinylpyrrolidone (PVP), polyvinyl alcohol (PVA), and the like; acrylic polymers and copolymers, such as acrylic acid polymer, methacrylic acid copolymers, ethyl acrylate-methyl methacrylate copolymers, and the like; and mixtures thereof. Currently, the preferred polymers include ethylcellulose and HPMC.

Fats and waxes that may be used in accordance with embodiments of the present invention include, but are not limited to, bees wax, carnuba wax, spermaceti, etc. Synthetic waxes include, but are not limited to, mineral oil, paraffin, microcrystalline wax, and polyethylene wax.

The amount of taste-masking agent that may be present varies from about 0.1% to about 50%. In some aspects, the taste-masking agent may range from about 1% to about 30%; about 1% to about 40%; about 5% to about 30%; about 5% to about 40%; about 10% to about 3.0%; about 0.1% to about 20%; about 0.1% to about 10%; about 1% to about 20%; about 1% to about 10%; about 5% to about 25%; about 5% to about 15%; or about 5% to about 10%.

It is understood that compounds described above may be considered applicable in more than more category. As such, the embodiments of the present invention may include compounds that can function in two or more capacities.

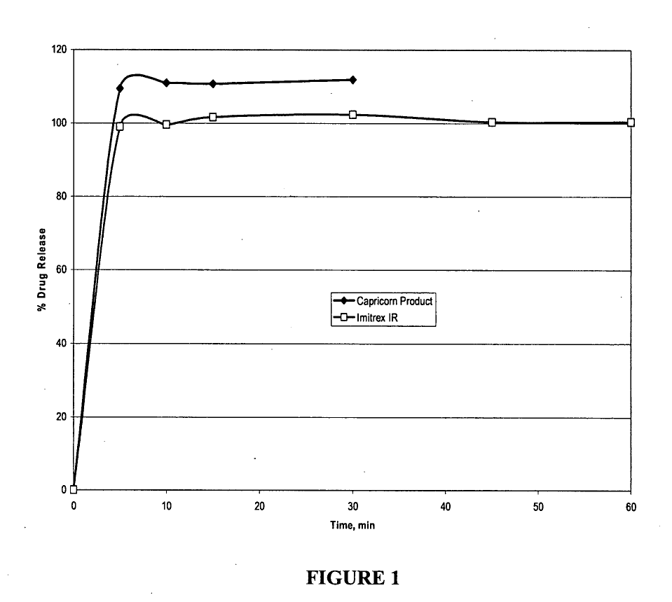

In another aspect, as illustrated in FIGS. 1-3, the triptan fast disintegrating composition of this invention has a dissolution profile that is comparable to the triptan dissolution profile obtained from an alternative oral dosage form, such as a tablet, capsule or a caplet, etc. of an equivalent dosage of the active ingredient. Thus, the compositions are taste-masked, disintegrate rapidly, and display a dissolution profile that is comparable to an alternate oral dosage form of the same active drug in the same strength.

In yet another aspect, the fast disintegrating compositions of this invention show, upon one month stability study under room temperature conditions as well as accelerated conditions (these conditions are known in the art, for example, at 50° C.) no changes in taste, tablet hardness, mouth dispersion or release at any pH.

The compositions described herein can be taste-masked through a resin forming a taste-masked composition and with an additional taste-masking agent as previously described. As such, the triptan composition can have a core of a triptan and resin further overcoated with a taste-masking agent. Alternately, the taste-masking agent can be homogenously admixed with the resin and triptan forming a substantially homogenous taste-masked triptan composition. Additionally, such triptan composition may contain lubricants, disintegrants, compressible materials, polymers, waxes, emulsifiers, fats, colorants, flavors, sweeteners, plasticizers, binding agents, filling agents, suspending agents, preservatives, buffers, wetting agents, effervescent agents, and other excipients as is known in the art. In one embodiment, the triptan compositions disclosed herein can be manufactured from pharmaceutically acceptable materials. In another embodiment, the taste-masked triptan compositions can be directly compressible.

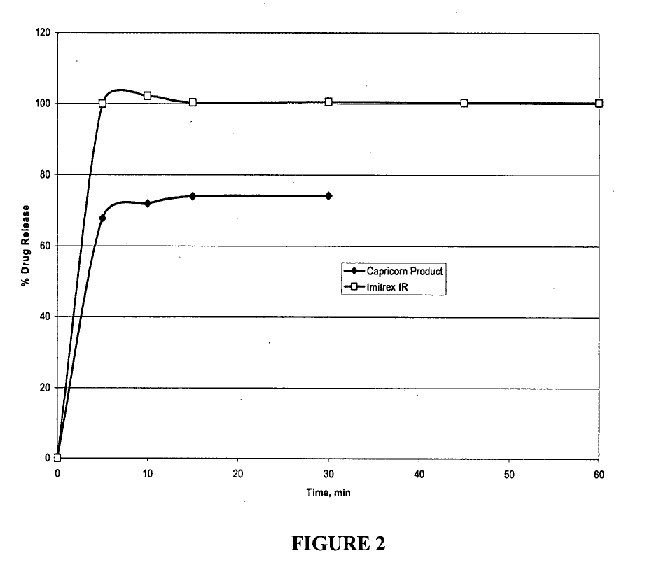

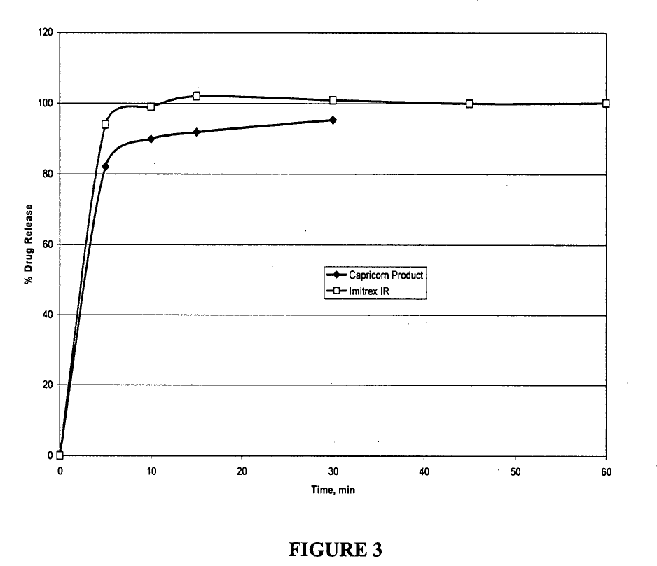

In one aspect, Sumatriptan Succinate was successfully taste-masked at a level of 20% drug loading (as Sumatriptan). The resulting powder had an acceptable taste with no hint of the sour/bitter flavor associated with Sumatriptan Succinate and a dissolution profile tested at a pH of 1.2, shown in FIG. 1, at a pH of 4.5, shown in FIG. 2, and at a pH of 6.8, shown in FIG. 3, comparable to that of IMITREX 25 mg immediate release tablets. Thus, the resulting powder was suitable to evaluate in a model tablet formulation.

Using the taste-masked product prepared above, 25 mg Sumatriptan (35 mg Sumatriptan Succinate) fast-disintegrating tablets were prepared. The total weight was 300 mg and the tablet hardness ranged from 2.5-3.0 kp. The tablets had good dispersion in the mouth (less than 25 seconds). The tablets also had a good taste comparable to that of the taste-masked powder indicating the suitability and robustness of the taste-masked powder for tabletting.

In vitro release of the Sumatriptan fast-disintegrating tablet was measured in three media, pH 1.2 (FIG. 1), 4.5 (FIG. 2), and 6.8 (FIG. 3) buffered solutions, and compared to that of IMITREX 25 mg immediate release tablets. FIGS. 1-3 show comparable dissolution. Thus, in one aspect, the fast-disintegrating triptan compositions of the present invention provide a dissolution profile that matches with an immediate release formulation of the same strength of the active when such dissolution is performed under USP conditions and at a pH of either 1.2 or 4.5 or 6.8.

It is to be understood that while the invention has been described in conjunction with the preferred specific embodiments thereof, that the description above as well as the examples which follow below are intended to illustrate and not limit the scope of the invention. Any modification within the scope of the invention will be apparent to those skilled in the art to which the invention pertains.

### EXAMPLES

The following examples are given to illustrate the present invention. It should be understood, however, that the invention is not to be limited to the specific conditions or details described in these examples.

### Example 1

### Taste-Masked

TABLE 1

| | DW |
|---|---|
| Material Name | Qty. % |
| Amberlite IRP 88** | 80.00 |
| Sumatriptan Succinate | 20.00 |
| Purified water | |
| Total | 100.00 |

The composition of Table 1 was used. Sumatriptan Succinate was weighed and dissolved in purified water. Amberlite IRP 88 was weighed and added to the drug solution and mixed thoroughly for about 6 hrs. The drug resin suspension is filtered and the wet cake mix is separated, dried in a suitable dryer at 50° C. for over 8 hours until the mixture's moisture content was reduced to about 8-12%. The drying can also be accomplished using fluid bed dryer for an hour.

Similarly, naratriptan, eletriptan, frovatriptan compositions are prepared. Similarly, other resins are used to prepare taste-masked triptan compositions. Additional examples were prepared using different coating % of the resin. These are shown in Table 1 a below.

TABLE 1a

| | DW | DW | DW | DW | DW |
|---|---|---|---|---|---|
| | Qty. % | Qty. % | Qty. % | Qty. % | Qty. % |
| Material | Example | Example | Example | Example | Example |
| Name | 1a | 1b | 1c | 1d | 1e |
| Amberlite | 75.00 | 70.00 | 65.00 | 60.00 | 55.00 |
| IRP 88** | | | | | |
| Sumatriptan | 25.00 | 30.00 | 35.00 | 40.00 | 45.00 |
| Succinate | | | | | |
| Purified | 100.00 | 100.00 | 100.00 | 100.00 | 100.00 |
| water QS to | | | | | |
| Total | | | | | |

### Example 2

### Tabletting

TABLE 2

| Material Name | DW Qty. % |
|---|---|
| Active Drug | |
| Taste-masked Sumatriptan | 54.14 |
| Succinate | |
| Excipients | |
| Microcrystalline cellulose powder | 29.28 |
| Lactittol Powder | 5.00 |
| Crospovidone | 7.00 |
| Cross caramellose Sodium | 1.00 |
| Amberlite IRP 64 | 2.00 |
| sodium Carbonate | 1.00 |
| Magnesium Stearate | 0.50 |
| Glycerine | Q.S. |
| Art masking flavor | Q.S |
| Total | 100.00 |

Tabletting was performed using the composition of Table 2. Dispersible taste-masked powder and other pharmaceutical acceptable ingredients, such as flavor, sweetener, colorant, and additional disintegrant in sufficient quantity to provide a therapeutically effective unit dose 25 mg sumatriptan in a V-blender for a sufficient time to get homogeneously distributed blending for compression. Unit dose was measured on an analytical balance and compressed into tablet at an optimized compression force on a tablet press.

### Example 3

### Granulation of Blended Sumatriptan

Blended sumatriptan from Example 2 is granulated using wet granulation procedures known in the art. In one aspect, the solvent is aqueous. Alternatively, solvents such as isopropanol or alcohol may be used. When nonaqueous solvents are used, emulsifiers, fats, waxes or combinations thereof may be used. These materials may be incorporated via hot melt spray coating, or solution spray granulation system whereby the solvent is removed. Examples of emulsifiers include acetylated monoglycerides, mono- and di-glyceride esters. Waxes may include synthetic and natural and combinations thereof. These granules are blended with other excipients and compressed into a tablet.

### Example 4

### Disintegration and Dissolution

Disintegration was measured using a stop clock and observing the disintegration in vitro as well as in vivo with volunteers. Dissolution Testing: Both microcapsules and tablets were tested for dissolution using USP Apparatus 2 (paddles@50 rpm) in 900 mL medium at 37° C. and percentage of drug released was determined by HPLC.

### Example 5

### Tabletting

TABLE 3

| Material Name | DW Qty. % |
|---|---|
| Active Drug | |
| Taste-masked Sumatriptan | 50.00 |
| Succinate20.00% | |
| Excipients | 0.00 |
| Microcrystalline cellulose powder | 20.27 |
| Mannitol granules | 20.27 |
| Crospovidone | 2.57 |
| Cross caramellose Sodium | 0.86 |
| Magnesium Stearate | 0.03 |
| Sucralose Powder | 1.21 |
| Art.masking flavor | Q.S. |
| Total | 100.00 |

Example 2 process was followed to make tablets of taste-masked sumatriptan.

### Example 6

### Tabletting

TABLE 4

| Material Name | DW Qty. % |
|---|---|
| Active Drug | |
| Taste-masked Sumatriptan | 46.67 |
| Succinate25.00% | |
| Excipients | |
| Microcrystalline cellulose powder | 42.29 |
| Crospovidone | 3.00 |
| Cross caramellose Sodium | 1.00 |
| Magnesium Stearate | 0.04 |
| Micronised Sucralose Powder | 2.00 |
| Art.masking flavor | Q.S |
| Total | 100.00 |

Example 2 process was followed to make tablets of taste-masked sumatriptan.

### Example 7

### Tabletting

TABLE 5

| Material Name | DW Qty. % |
|---|---|
| Active Drug | |
| Taste-masked Sumatriptan Succinate | 72.90 |
| 25.00% | |
| Excipients | |
| Mannitol granules | 17.57 |
| Crospovidone | 3.00 |
| Cross caramellose Sodium | 1.00 |
| Magnesium Stearate | 0.40 |
| Purified talc | 0.13 |
| Micronised Sucralose Powder | 2.00 |
| Art.masking flavor | Q.S. |
| Total | 100.00 |

### Example 2 process was followed to make tablets of taste-masked sumatriptan.

### Example 8

### Tabletting

TABLE 6

| Material Name | DW Qty. % |
|---|---|
| Active Drug | |
| Taste-masked Sumatriptan | 18.35 |
| Succinate 97.56% | |
| Microcrystalline cellulose | 56.85 |
| Excipients | |
| Crospovidone (Polyplasdone | 5.00 |
| XL10) | |
| Cross caramellose Sodium | 1.00 |
| Magnesium Stearate | 0.60 |
| Purified talc | 0.20 |
| Silica (Syloid 200 FP) | 1.00 |
| Micronised Sucralose Powder | 5.00 |
| Glycine | 3.00 |
| Art.masking flavor | Q.S. |
| Total | 100.00 |

In this Example, resin-taste-masked triptan is further coated with microcrystalline cellulose and then tabletted. Example 2 process was followed to make tablets of taste-masked sumatriptan.

What is claimed is:

1. A rapidly disintegrating oral triptan composition, comprising:
a) a triptan compound,
b) a resin,
c) a lubricant,
d) a disintegrant, and
e) a compressible material,
wherein the triptan is admixed with the resin forming a taste-masked triptan composition, said taste-masked triptan composition further admixed with the lubricant, the disintegrant, and the compressible material to form a rapidly disintegrating oral triptan composition.

2. The composition of claim 1, wherein the triptan compound is selected from the group consisting of sumatriptan, eletriptan, rizatriptan, frovatriptan, almotriptan, zolmitriptan, naratriptan, pharmaceutically acceptable salt or hydrate thereof, and mixtures thereof.

3. The composition of claim 1, wherein the triptan compound is present in an amount from about 0.1 wt % to about 50 wt %.

4. The composition of claim 1, wherein the resin is selected from the group consisting of cholestyramine resin, cationic resins including copolymers of methacrylic acid crosslinked with divinylbenzene, sodium polystyrene sulfonate, polacrilin potassium and mixtures thereof.

5. The composition of claim 1, wherein the resin is present in an amount from about 20 wt % to about 95 wt %.

6. The composition of claim 1, wherein the lubricant is selected from the group consisting of magnesium stearate, zinc stearate, calcium stearate, stearic acid, glyceryl palmitostearate, vegetable oils, polyethylene glycols, polyvinyl alcohols, talc, sodium benzoate, sodium stearyl fumarate, magnesium oxide, poloxamer, sodium lauryl sulphate, polyoxy ethylene monostearate, cocoa butter, hydrogenated tallow, hydrogenated cotton seed oil, canola oil, palm kernel oil, soybean oil, stannol esters, hydrogenated palm kernel oil, mineral oil, cocoa butter, cocoa butter substitutes, polysaccharides, and mixtures thereof.

7. The composition of claim 1, wherein the lubricant is present in an amount from about 0.1 wt % to about 30 wt %.

8. The composition of claim 1, wherein the disintegrant is selected from the group consisting of mannitol, alginic acid, carboxymethylcellulose, hydroxypropylcellulose, microcrystalline cellulose, colloidal silicon dioxide, croscarmellose sodium, crospovidone, magnesium aluminum silicate, methylcellulose, povidone, sodium alginate, sodium starch glycolate, and mixtures thereof.

9. The composition of claim 1, wherein the disintegrant is present in an amount from about 1 wt % to about 75 wt %.

10. The composition of claim 1, wherein the compressible material is selected from the group consisting of monosaccharides, disaccharides, oligosaccharides, polysaccharides, arabitol, erythritol, hydrogenated starch hydrolysates, isomalt, lactitol, maltitol, mannitol, sorbitol, xylitol, galactitol, inositol, ribitol, dithioerythritol, dithiothreitol, glycerol, calcium phosphates, microcrystalline celluloses, polydextrose, erythritols, and mixtures thereof.

11. The composition of claim 1, wherein the compressible material is present in an amount from about 1 wt % to about 75 wt %.

12. The composition of claim 1, further comprising a taste-masking agent selected from the group consisting of a polymer, a fat, a wax, an emulsifier, and mixtures thereof.

13. The composition of claim 12, wherein the taste-masking agent is present in amount from about 0.1 wt % to about 50 wt %.

14. The composition of claim 12, wherein the polymer is selected from the group consisting of cellulosic polymers including methylcellulose (MC), carboxymethylcellulose (CMC), hydroxypropylcellulose (HPC), hydroxylpropylmethylcellulose (HPMC), hydroxyethylcellulose (HEC); vinyl polymers including polyvinylpyrrolidone (PVP), polyvinyl alcohol (PVA); acrylic polymers and copolymers including acrylic acid polymer, methacrylic acid copolymers, ethyl acrylate-methyl methacrylate copolymers; and mixtures thereof.

15. The composition of claim 12, wherein the emulsifier is selected from the group consisting of alkyl aryl sulfonates, alkyl sulfates, sulfonated amides and amines, sulfated and sulfonated esters and ethers, alkyl sulfonates, polyethoxylated esters, monoglycerides, diglycerides, triglycerides, diacetyl tartaric esters of monoglycerides, polyethyleneglycol esters, polyglycerol esters, sorbitan esters and ethoxylates, lactylated esters, phospholipids such as lecithin, polyoxyethylene sorbitan esters, propylene glycol esters, sucrose esters, and mixtures thereof.

16. The composition of claim 12, wherein the fat and/or wax is individually selected from the group consisting of bees wax, carnuba wax, spermaceti, mineral oil, paraffin, microcrystalline wax, polyethylene wax and mixtures thereof.

17. The composition of claim 12, wherein the taste-masked composition forms a core and the taste-masking agent forms an overcoat.

18. The composition of claim 12, wherein the taste-masking agent is homogenously admixed in the rapidly disintegrating oral triptan composition.

19. The composition of claim 1, wherein the composition disintegrates in an oral cavity in less than about 60 seconds.

20. The composition of claim 1, wherein the composition disintegrates in an oral cavity in less than about 45 seconds.

21. The composition of claim 1, wherein the composition disintegrates in an oral cavity in less than about 30 seconds.

22. The composition of claim 1, wherein the composition is formulated from pharmaceutically acceptable materials.

23. The composition of claim 1, wherein the taste-masked triptan is directly compressible.

24. A method of making a rapidly disintegrating oral triptan composition, comprising:
a) admixing a resin and a triptan compound forming a taste-masked triptan compound; and
b) admixing the taste-masked triptan compound with a lubricant, a disintegrant, and a compressible material forming a rapidly disintegrating oral triptan composition.

25. The method of claim 24, wherein the triptan compound is selected from the group consisting of sumatriptan, eletriptan, rizatriptan, frovatriptan, almotriptan, zolmitriptan, naratriptan, pharmaceutically acceptable salt or hydrate thereof, and mixtures thereof.

26. The method of claim 24, wherein the triptan compound is present in an amount from about 0.1 wt % to about 50 wt %.

27. The method of claim 24, wherein the resin is selected from the group consisting of cholestyramine resin, cationic resins including copolymers of methacrylic acid crosslinked with divinylbenzene, sodium polystyrene sulfonate, polacrilin potassium and mixtures thereof.

28. The method of claim 24, wherein the resin is present in an amount from about 20 wt % to about 95 wt %.

29. The method of claim 24, wherein the lubricant is selected from the group consisting of magnesium stearate, zinc stearate, calcium stearate, stearic acid, glyceryl palmitostearate, vegetable oils, polyethylene glycols, polyvinyl alcohols, talc, sodium benzoate, sodium stearyl fumarate, magnesium oxide, poloxamer, sodium lauryl sulphate, polyoxy ethylene monostearate, cocoa butter, hydrogenated tallow, hydrogenated cotton seed oil, canola oil, palm kernel oil, soybean oil, stannol esters, hydrogenated palm kernel oil, mineral oil, cocoa butter, cocoa butter substitutes, polysaccharides, and mixtures thereof.

30. The method of claim 24, wherein the lubricant is present in an amount from about 0.1 wt % to about 30 wt %.

31. The method of claim 24, wherein the disintegrant is selected from the group consisting of mannitol, alginic acid, carboxymethylcellulose, hydroxypropylcellulose, microcrystalline cellulose, colloidal silicon dioxide, croscarmellose sodium, crospovidone, magnesium aluminum silicate, methylcellulose, povidone, sodium alginate, sodium starch glycolate, and mixtures thereof.

32. The method of claim 24, wherein the disintegrant is present in an amount from about 1 wt % to about 75 wt %.

33. The method of claim 24, wherein the compressible material is selected from the group consisting of monosaccharides, disaccharides, oligosaccharides, polysaccharides, arabitol, erythritol, hydrogenated starch hydrolysates, isomalt, lactitol, maltitol, mannitol, sorbitol, xylitol, galactitol, inositol, ribitol, dithioerythritol, dithiothreitol, glycerol, calcium phosphates, microcrystalline celluloses, polydextrose, erythritols, and mixtures thereof.

34. The method of claim 24, wherein the compressible material is present in an amount from about 1 wt % to about 75 wt %.

35. The method of claim 24, further admixing a taste-masking agent selected from the group consisting of a polymer, a fat, a wax, an emulsifier, and mixtures thereof.

36. The method of claim 35, wherein the taste-masking agent is present in amount from about 0.1 wt % to about 30 wt %.

37. The method of claim 35, wherein the taste-masked composition forms a core and the taste-masking agent forms an overcoat.

38. The method of claim 35, wherein the taste-masking agent is homogenously admixed in the rapidly disintegrating oral triptan composition.

39. A method of administering a triptan composition to a subject, comprising:
a) providing the triptan composition in an rapidly disintegrating oral dosage form, said rapidly disintegrating oral comprising:
a) a triptan compound,
b) a resin,
c) a lubricant,
d) a disintegrant, and
e) a compressible material,
wherein the triptan is admixed with the resin forming a taste-masked triptan composition, said taste-masked triptan composition further admixed with the lubricant, the disintegrant, and the compressible material to form a rapidly disintegrating oral triptan composition, and
b) administering the rapidly disintegrating oral dosage form to the subject's oral cavity.

40. The method of claim 39, wherein the triptan compound is selected from the group consisting of sumatriptan, eletriptan, rizatriptan, frovatriptan, almotriptan, zolmitriptan, naratriptan, pharmaceutically acceptable salt or hydrate thereof, and mixtures thereof.

41. The method of claim 39, wherein the triptan compound is present in an amount from about 0.1 wt % to about 50 wt %.

42. The method of claim 39, wherein the resin is selected from the group consisting of cholestyramine resin, cationic resins including copolymers of methacrylic acid crosslinked with divinylbenzene, sodium polystyrene sulfonate, polacrilin potassium, and mixtures thereof.

43. The method of claim 39, wherein the resin is present in an amount from about 20 wt % to about 95 wt %.

44. The method of claim 39, wherein the lubricant is selected from the group consisting of magnesium stearate, zinc stearate, calcium stearate, stearic acid, glyceryl palmitostearate, vegetable oils, polyethylene glycols, polyvinyl alcohols, talc, sodium benzoate, sodium stearyl fumarate, magnesium oxide, poloxamer, sodium lauryl sulphate, polyoxy ethylene monostearate, cocoa butter, hydrogenated tallow, hydrogenated cotton seed oil, canola oil, palm kernel oil, soybean oil, stannol esters, hydrogenated palm kernel oil, mineral oil, cocoa butter, cocoa butter substitutes, polysaccharides, and mixtures thereof.

45. The method of claim 39, wherein the lubricant is present in an amount from about 0.1 wt % to about 30 wt %.

46. The method of claim 39, wherein the disintegrant is selected from the group consisting of mannitol, alginic acid, carboxymethylcellulose, hydroxypropylcellulose, microcrystalline cellulose, colloidal silicon dioxide, croscarmellose sodium, crospovidone, magnesium aluminum silicate, methylcellulose, povidone, sodium alginate, sodium starch glycolate, and mixtures thereof.

47. The method of claim 39, wherein the disintegrant is present in an amount from about 1 wt % to about 75 wt %.

48. The method of claim 39, wherein the compressible material is selected from the group consisting of monosaccharides, disaccharides, oligosaccharides, polysaccharides, arabitol, erythritol, hydrogenated starch hydrolysates, isomalt, lactitol, maltitol, mannitol, sorbitol, xylitol, galactitol, inositol, ribitol, dithioerythritol, dithiothreitol, glycerol, calcium phosphates, microcrystalline celluloses, polydextrose, erythritols, and mixtures thereof.

49. The method of claim 39, wherein the compressible material is present in an amount from about 1 wt % to about 75 wt %.

50. The method of claim 39, further admixing a taste-masking agent selected from the group consisting of a polymer, a fat, a wax, an emulsifier, and mixtures thereof.

51. The method of claim 50, wherein the taste-masking agent is present in amount from about 0.1 wt % to about 30 wt %.

52. The method of claim 50, wherein the taste-masked composition forms a core and the taste-masking agent forms an overcoat.

53. The method of claim 50, wherein the taste-masking agent is homogenously admixed in the rapidly disintegrating oral triptan composition.

54. A method of masking an unpleasant taste of a triptan active agent to be administered to the oral cavity of a subject comprising:
combining the triptan active agent with a resin into an oral dosage formulation.
